(19) United States
(12) Patent Application Publication
(10) Pub. No.: US 2024/0156629 A1
(43) Pub. Date: May 16, 2024

(54) FOOT ORTHOSIS FOR CORRECTING FOOT MALPOSITIONS HAVING A TOE SEGMENT IN THE FORM OF A BRACKET

(71) Applicant: Elsa Vermögens- und Beteiligungs AG, Grünwald (DE)

(72) Inventors: Manfred BRASS, Grünwald (DE); Jörg OSTENRIEDER, Ebenhausen (DE)

(73) Assignee: Elsa Vermögens- und Beteiligungs AG, Grünwald (DE)

(21) Appl. No.: 18/551,742

(22) PCT Filed: Mar. 22, 2022
(86) PCT No.: PCT/EP2022/057523

§ 371 Date: Sep. 21, 2023

(30) Foreign Application Priority Data
Mar. 22, 2021 (DE) 10 2021 107 082.1

(51) Int. Cl. A61F 5/01

(52) U.S. Cl. CPC A61F 5/019

(57) ABSTRACT

A foot orthosis for correcting foot malpositions, in particular for treating hallux valgus, comprising a toe segment configured to be fastened to a toe and a ball segment configured to be arranged in the region of a metatarsophalangeal joint which are pivotably connected relative to one another by means of a joint unit. When the foot orthosis is property fastened to the foot, the foot orthosis is configured to exert a first corrective force on the toe via the toe segment and to exert a second corrective force on the metatarsophalangeal joint via the ball segment in the opposite direction to the first corrective force. The toe segment is provided in the form of a bracket which, in the fastened state, at least partially engages around the toe.

### CROSS REFERENCE TO RELATED APPLICATIONS

This application is a national stage U.S. patent application of International Application No. PCT/EP20221057523, filed on Mar. 22, 2022, and claims foreign priority to German Patent Application No. DE 10 2021 107 082.1, filed on Mar. 22, 2021, the entirety of each of which is incorporated herein by reference.

### TECHNICAL FIELD

A foot orthosis for correcting malpositions of a foot, in particular for treating hallux valgus.

### TECHNOLOGICAL BACKGROUND

Pathological malpositions in the metatarsal and forefoot area of a patient can have various causes, such as genetic predisposition, wearing wrong footwear, especially shoes that are too tight or high-heeled, or a flattening of the longitudinal and transverse arch as a result of instability of the connective tissue in the metatarsal area. In particular, malpositioning of the big toe in the metatarsophalangeal joint, also known as hallux valgus, is gaining in importance due to steadily increasing number of cases.

Hallux valgus emerges from the metatarsophalangeal joint of the big toe being pulled in the direction of the inside of the foot by muscle traction. This causes the first metatarsal to protrude from the inside of the foot as a ban-shaped protrusion at the metatarsophalangeal joint, which is referred to as pseudoexostosis. In addition, hallux valgus is often accompanied by a change in the length and direction of traction of tendons, which can further exacerbate the deformly over time. As a result, arthrosis of the metatarsophalangeal joint of the big toe develops, which has to be treated surgically in advanced stages.

To stop or counteract the disease process, in addition to surgical interventions, the use of conservative therapy methods is known. For example, the use of tape bandages or orthoses is known for treating the foot in a resting position. Due to the required resting position of the foot during therapy, these are mainly used at night.

Furthermore, orthoses are known which, in a state fastened to the foot, allow a splinted big toe to move along its flexion-extension direction. For example, DE 102 40 121 B4 discloses an orthopedic device in the form of a hinged bending splint which is articulated about a flexion-extension movement axis of a toe to be corrected. For this purpose, the hinged bending splint is provided with a joint resting against the inner side of the foot and with two bending legs extending from the joint along the inner side of the foot. For fastening the hinged bending splint to the foot, a first bending leg is fastened to the toe via a first bandage and a second bending leg is fastened to the metatarsal via a second bandage.

### SUMMARY

The present disclosure describes a foot orthosis for correcting malpositions, in particular for treating hallux valgus, which in particular ensures effective therapeutic treatment and at the same time can be easily manufactured and has a compact design.

A foot orthosis is provided for correcting foot malpostions, in particular for treating or preventing hallux valgus. The foot orthosis comprises a toe segment configured for being fastened to a toe of a foot to be treated and a ball segment configured for being arranged in the region of a metatarsophalangeal joint of the foot to be treated which are pivotably connected relative to one another by means of a joint unit. The foot orthosis is designed and configured, in a fastened state in which the foot orthosis is property fastened to the foot, to exert a first corrective force on the toe via the toe segment and to exert a second corrective force on the metatarsophalangeal joint via the ball segment in the opposite direction to the first corrective. The toe segment is provided in the form of a bracket or clamp which, in the fastened state, at least partially engages around the toe.

For interacting with the toe of the foot to be treated, i.e. for exerting the first corrective force on the toe, the foot orthosis is equipped with the toe segment which is designed as a bracket or clamp engaging around the toe. In the context of the present disclosure, t has been found that, by designing the toe segment in the form of a bracket or clamp which at least partially engages around the toe, a force-flow- and load-optimized structural design may be provided for the intended application of the foot orthosis. This is because the bracket- or clamp-like toe segment, which at least partially engages around the toe, enables that the thus resulting structure, at least in sections, may be provided with an increased stiffness, in particular with an increased geometrical moment of inertia, with respect to bending forces transmitted in the toe segment for inducing the first corrective force. In this way, the toe segment can be designed, at least in sections, narrower and/or thinner and accordingly may contribute to a compact design of the foot orthosis. Wearing the foot orthosis in conventional footwear may thus be significantly more comfortable for the patient compared to known devices having bending legs which extend along the toe on the foot inner side. With this arrangement, the described solution further enables that bandages for fastening the foot orthosis to the foot may be omitted. Accordingly, the foot orthosis may comprise less components and therefore may be produced at lower costs.

The foot orthosis is intended and configured for treating, counteracting and/or preventing pathological foot malpositions, in particular malpositions of a toe and/or its metatarsophalangeal joint. In particular, the foot orthosis may be used for preventing or treating hallux valgus, but is not limited to this application. Accordingly, the foot orthosis is intended and configured to be fastened to a patient's foot and, in the state fastened to the foot, to act therapeutically on the foot, in particular on the toe and/or the metatarsophalangeal joint.

In the present disclosure, the term “in a/the state property fastened to the foot”, herein also referred to as “in the fastened state”, refers to a state in which the foot orthosis is property fastened to a patient's foot and accordingly produces a desired therapeutic effect for correcting or preventing malpositions. The foot orthosis may be foot-specifically designed for a left or right foot of a patient.

In other words, the foot orthosis may be intended and designed for use on either the left or right foot of the patient. A foot orthosis intended for the left foot may be mirror symmetric to a foot orthosis intended for the right foot of a patient.

The foot orthosis is designed to, in the fastened state, exert corrective forces on the foot. In the present disclosure, the term “corrective forces” refers to forces that have a therapeutic effect on the foot to be treated. In particular, the corrective forces cause those parts of the foot affected by the malposition to be positioned into or towards an anatomically correct or intended position to achieve a desired therapeutic effect.

The foot orthosis is configured to, in the fastened state on the patient's foot, exert, in particular directly exert, at least the first and the second corrective force on the foot to be treated by means of the toe segment and the ball segment. By this design, the present foot orthosis differs substantially from known devices which, intentionally or unintentionally, shield a metatarsophalangeal joint and the ball of the toe associated therewith or a pathological pseudoexostosis caused by the malposition from external forces, in particular from forces acting on the foot through the device. In the context of the present disclosure, t has been found that a particularly effective therapeutic effect can be achieved if the foot orthosis, in addition to the first corrective force acting on the toe, also exerts the second corrective force acting on the metatarsophalangeal joint by means of the ball segment. The resulting interaction of corrective forces exerted on the foot can be particularly beneficial in the treatment of hallux valgus. This is because the embodiment of the foot orthosis can have a simultaneous therapeutic effect on the valgus position of the toe and on the varus position of the metatarsophalangeal joint. By doing so, the symptoms and the cause of the foot malposition can be treated simultaneously. The corrective forces acting on the foot and the associated therapeutic effects are described in more detail below in connection with the related components of the foot orthosis.

In the following, in connection with the toe segment, it is generally referred to a toe of the foot for the sake of simplicity which means the big toe of the foot to be treated. However, the foot orthosis is not limited to this application such that the term “toe” may also relate to, for example, the little toe. In connection with the ball segment, accordingly, t is generally referred to a metatarsophalangeal joint which means the metatarsophalangeal joint of the big toe. Yet, the foot orthosis is not limited to this application. Alternatively, the term metatarsophalangeal joint may refer to, for example, the metatarsophalangeal joint of the little toe.

In the present disclosure, for specifying the foot orthosis, in particular with respect to the foot to be treated, a reference system is used which is oriented to the midline or medial plane of a patient's body, as is common in anatomy. Thus, the position and direction of each component of the foot orthosis in the fastened state may be indicated with respect to the foot received in the foot orthosis. Accordingly, the term “medial” refers to a direction or side of the foot orthosis that points toward a medial plane of the wearer's body. In anatomy, the term “medial plane”, also known as “mid-sagittal plane”, generally refers to an anatomical plane that divides the body into two symmetrical parts. Accordingly, when describing a foot orthosis, the term “in medial direction” means a direction pointing from the patient's foot to be treated towards his other foot. In this sense, the term “lateral” refers to a direction or side of the foot orthosis that points away from the medial plane of the wearer's body. Accordingly, when describing a foot orthosis fastened to one foot of the wearer, the term “lateral” means in a direction facing away from the other foot of the wearer.

The foot orthosis includes the toe segment configured to be fastened to the toe of the foot to be treated. In other words, the toe segment is intended and configured to be brought into engagement with the toe of the foot in a predefined position to provide a force-transmitting coupling between the toe and the toe segment in the fastened state. Accordingly, in the fastened state, the toe segment is held in a desired position and in particular is force- and/or form-fittingly fastened to the toe.

In the fastened state, the toe segment is intended and configured to exert the first corrective force on the toe. The first corrective force may act on the toe in the medial direction.

As described above, the toe segment is provided in the form of a bracket or clamp. In the context of the present disclosure and generally, the term “bracket” or “clamp” refers to a component that is designed and provided to receive and transmit different loads, such as longitudinal forces, transverse forces, shear forces, bending forces, bending moments, torsional moments, etc. As such, a bracket or clamp is configured to receive and transmit not only tensile forces but also compressive forces along its longitudinal axis and transverse forces transverse to its extension axis, in particular transvers to its longitudinal axis. This structural configuration substantially distinguishes a bracket or clamp from a bandage, which is provided for the transmission of tensile forces but not for the transmission of compressive and/or transverse forces.

Accordingly, the toe segment provided in the form of a clamp may be configured, in the fastened state, to receive and transmit shearing forces and/or bending forces, in particular in direction of the first corrective force to the foot and/or between the joint unit and the toe to be treated, in order to exert the first corrective force on the toe.

The toe segment provided in the form of a bracket is designed such that, in the fastened state, it at least partially engages around the toe, i.e. lies around the toe. In the context of the present disclosure, the term “engaging around the toe” means that, in the fastened state, the toe segment extends around the toe along its circumferential direction. The toe segment can extend along the toe over a radian of at least ½ π rad around the longitudinal axis of the toe. That is, the toe segment extends circumferentially along at least a quarter of the circumference of a toe. For example, the toe segment may extend over a radian of substantially one π rad about the longitudinal axis of the toe such that the toe segment extends from one side of the toe or the metatarsophalangeal joint to the opposite side of the toe.

With regard to the geometric design, the toe segment, provided in the form of a bracket, may be plate-shaped and/or shell-shaped. For example, with regard to its geometric design, the toe segment may be belt- or band-shaped and particularly may be bending resistant. Accordingly, the toe segment may be provided with a contact surface for the toe and an opposite support surface. In the fastened state of the foot orthosis, the contact surface of the toe segment may lie against the toe, in particular may touch the toe. The contact surface may be provided in the form of a turning surface, the orientation of which, i.e. whose surface normal, changes along the longitudinal axis of the toe and can point to the longitudinal axis of the toe. By this configuration, the contact surface and thus also the toe segment may extend along a helical line around the longitudinal axis of the toe.

To apply or exert the first corrective force to or onto the toe, the toe segment may comprise a toe support section which, in the fastened state of the foot orthosis, lies against the toe, in particular lies against a lateral side of the toe, i.e. against a side oriented in the lateral direction. In other words, the toe support section may be configured to apply or exert the first corrective force to or onto the toe, wherein the first corrective force points in or coincides with, in particular substantially coincides with, the medial direction. The toe support section may be provided by an end section of the toe segment, which in particular may be located distally.

The toe segment may further comprise a toe base section, which may form a section of the toe segment extending along the upper side or along the lower side of the toe to be treated. In other words, the toe base section may be arranged plantar and/or dorsal. Accordingly, in the fastened state of the foot orthosis, the toe base section may be parallel or substantially parallel to the natural bearing surface of the foot. According to one embodiment, in the fastened state, the toe may at least partially lie on the toe base section. Alternatively, the toe base section may, at least in sections, lie on the toe. The toe base section can be connected to the toe support section in a force-transmitting and/or torque-transmitting manner, for example integrally connected or adhesively bonded.

The toe support section may be provided in the form of a limb or may comprise a limb, and in particular may form a bending spring or spring element. In particular, the toe support section may be thin-walled. Furthermore, the toe support section may be arranged perpendicular or substantially perpendicular to the toe base section. Alternatively or additionally, the toe support section may be arranged vertically or substantially vertically.

The toe support section may be arranged laterally to the toe. Accordingly, the toe support section may directly exert the first corrective force on the toe by pressing on the toe h the medial direction. Alternatively, the toe support section may comprise or constitute a support band, in particular in the form of a strap, a loop, a band, a bell, etc. The support band may contact the lateral side of the toe and may exert the first corrective force thereon, wherein the support band may be provided or loaded with a corresponding tensile force at its end portions. For doing so, the support band may be fastened to a limb of the toe support section which may be arranged medially to the toe and/or the support band may be connected to the toe base section in a force-transmitting manner. In other words, in the fastened state, the toe may be arranged in the support band such that the support band at least partially lies against the toe in the circumferential direction. The support band may be tensile stiff or elastic, in particular along its circumferential direction.

The toe support section, in particular the toe base section and/or the toe support section, may be thin-walled, thereby contributing to a compact design of the foot orthosis. In particular, the toe segment, in particular the toe base section and/or the toe support section, may have a maximum wall thickness of less than 3 mm or less than 2 mm or less than 1 mm. In other words, the toe segment, in particular the toe base section and/or the toe support section, may be constructed from at least one plate-shaped component. Furthermore, the toe segment, in particular the toe base section and/or the toe support section, may be shell- or cup-shaped, and in particular may be adapted in its shape to the shape of the toe to be treated. In this way, a particularly large contact surface for the toe may be provided, thereby making it more comfortable for a patient to wear the foot orthosis. Further, a compact design of the foot orthosis may be ensured.

According to an embodiment, the toe segment may be provided in the form of a clamping bracket. By this configuration, the first corrective force can be provided in the form of a clamping force induced by an elastic deformation of the toe segment.

The foot orthosis may be configured such that, in a state decoupled from the foot, i.e. in a state in which the foot orthosis is not engaging with the foot to be treated and thus is detached from the foot, the foot orthosis, in particular the toe segment, is arranged in a rest position in which the foot orthosis is not elastically deformed. However, in the fastened state, the foot orthosis, in particular the toe segment, may be arranged in a clamping position in which the foot orthosis, in particular the toe segment, is elastically deformed. The toe segment, in particular the toe support section, may be elastically deflected in the clamping position relative to the rest position hi a direction opposite to the first corrective force. For example, in the clamping position, the toe support section, in particular an end section of the toe support section, may be deflected or translationally displaced relative to the rest position by at least 0.2 cm, e.g., by at least 0.3 cm or at least 0.5 cm or at least 1.0 cm, in particular along the direction opposite to the first corrective force.

In the clamping position, the toe segment, by its elastic deformation, may be loaded or pre-loaded in direction of the first corrective force. The clamping force or the bending force exerted on the toe segment, in particular on the toe support section, may correspond to the first corrective force.

Specifically, the absolute value of the clamping force or bending force exerted on the toe segment, in particular on the toe support section, may be substantially equal to the absolute value of the first corrective force.

The foot orthosis further comprises the ball segment configured for being arranged in the region of the metatarsophalangeal joint when the foot orthosis is fastened to the foot. In particular, the ball segment may be configured for contacting the foot n the region of the metatarsophalangeal joint. In other words, the ball segment is provided to be engaged with the foot in the region of the metatarsophalangeal joint to provide, in the fastened state, a force-transmitting coupling between the foot in the region of the metatarsophalangeal joint and the ball segment. Accordingly, in the fastened state, the ball segment is fastened to the foot, hi particular force- and/or form-fittingly fastened to the foot, and is thus held at a desired position on the foot.

In particular, the ball segment may be designed and configured such that, hi the fastened state, the ball segment lies against the side of the foot in the region of the metatarsophalangeal joint, in particular against the side of the ball of the metatarsophalangeal joint. For example, in the fastened state, the ball segment may be against or be arranged hi the region of a lateral, ball-shaped protrusion of the foot, in particular of the first metatarsal, which is also referred to as a pseudoexostosis. In an embodiment, the ball segment can be configured such that, in the fastened state, the ball segment lies against the foot in the region of the metatarsal ball, but does not lie against the metatarsal ball or a pseudoexostosis itself. For doing so, the ball segment may be provided with a recess or through hole which, in the fastened state on the foot, receives at least a section of the metatarsal ball, in particular the ban-shaped protrusion on the foot, e.g., the pseudoexostosis. A section of the ball segment delimiting the recess or the through hole may be against the foot in a region adjacent to the ball of the metatarsophalangeal joint, in particular adjacent to the ball-shaped protrusion on the foot, e.g., to the pseudoexostosis. In other words, the foot orthosis can be configured such that, in the fastened state, a sideways or laterally protruding section of the metatarsophalangeal ball is received in the recess or through hole of the ball segment. For example, on the foot, the ball segment may be arranged circumferentially around the ball of the metatarsophalangeal joint, in particular around the ban-shaped protrusion on the foot, for example around the pseudoexostosis. The recess or through hole of the ball segment may have a cross-sectional diameter, in particular a minimum or maximum diameter, of at least substantially 1.5 cm or of at least substantially 2.0 cm or of at least substantially 2.5 cm. This embodiment enables the foot orthosis to exert the second corrective force on the metatarsophalangeal joint by means of the ball segment without having to directly act on, exert forces on or touch pain-sensitive regions of the foot.

In the context of the present disclosure, the feature defining that the “second corrective force is exerted on the metatarsophalangeal joint” relates to such corrective forces which therapeutically act on the metatarsophalangeal joint. Such corrective forces may be introduced directly into the metatarsophalangeal joint by means of the foot orthosis. Alternatively, such corrective forces may act indirectly on the metatarsophalangeal joint by means of the foot orthosis, for example by the ball segment exerting a corrective force on a metatarsal, in particular the first metatarsal. The second corrective force particularly is intended to therapeutically act on the varus position of a metatarsal, in particular on the varus position of the first metatarsal.

For treating hallux valgus, the foot orthosis may be provided such that, in the fastened state, the ball segment lies against the metatarsophalangeal joint on a medial side of the foot. Accordingly, the second corrective force may act on the metatarsophalangeal joint in a lateral direction.

The ball segment may be configured to, in the fastened state, receive and transmit shearing forces and/or bending forces, in particular in direction of the second corrective force and/or between the joint unit and the metatarsophalangeal joint to be treated, to exert the second corrective force on the metatarsophalangeal joint. In particular, the ball segment can be provided in the form of a bracket or clamp or may constitute a section of a bracket or clamp. According to an embodiment, the ball segment may at least partially engage around the ball of the metatarsophalangeal joint. For doing so, the ball segment may extend from a lateral side of the foot in the region of the metatarsophalangeal joint in direction of the sole of the foot and may in particular be adapted in its shape to the shape of the arch of the foot. Alternatively, the ball segment may extend from the lateral side of the foot in direction of the dorsum of the foot. For example, the ball segment may extend around the ball of the metatarsophalangeal joint over a radian of at least ½ π rad around the longitudinal axis of the toe.

With regard to the geometric design, the ball segment can be configured to be plate-shaped or shell-shaped. For example, with regard to its geometric design, the ball segment may be bet- or band-shaped. The ball segment can be bending resistant. Accordingly, the ball segment may be provided with a contact surface for the foot and an opposite contact surface. In the fastened state, the contact surface of the ball segment may be against the foot, in particular may touch the foot.

The contact surface may be provided in the form of a turning surface whose orientation, i.e. whose surface normal, changes along a longitudinal axis substantially parallel to the longitudinal axis of the toe and can point to the longitudinal axis. By this configuration, the contact surface and thus also the ball segment may extend along a helical line around the longitudinal axis.

To apply or exert the second corrective force to or onto the foot, the ball segment may comprise a ball support section which, in the fastened state, may lie against the ball of the metatarsophalangeal joint, in particular against a medial side of the foot. In other words, the ball support section may be configured to exert the second corrective force on the metatarsophalangeal joint, wherein the second corrective force points in the direction of or coincides with, in particular substantially coincides with, the lateral direction. The ball support section may be provided by an end section of the ball segment.

The ball segment may further comprise a ball base section, which may form a section of the ball segment extending along the sole or along the dorsum of the foot to be treated. In other words, the ball base section may be arranged plantar and/or dorsal. According to an embodiment, in the fastened state, the ball base section may be parallel or substantially parallel to the natural bearing surface of the foot. According to an embodiment, in the fastened state, the ball of the metatarsophalangeal joint may at least partially lie on the ball base section. The ball base section can be connected to the ball support section in a force-transmitting and/or torque-transmitting manner, for example integrally connected or adhesively bonded.

The ball support section can be provided in the form of a limb or may comprise a limb, and in particular may form a bending spring. In particular, the ball support section may be thin-walled.

Furthermore, the ball support section can be arranged vertically. According to one embodiment, the ball support section may be arranged perpendicular or substantially perpendicular to the ball base section. Alternatively or additionally, the ball support section may be arranged vertically or substantially vertically.

The ball segment, in particular the ball base section and/or the ball support section, may be thin-walled, thereby contributing to a compact design of the foot orthosis. In particular, the ball segment, in particular the ball base section and/or the ball support section, may have a maximum wall thickness of less than 3 mm or less than 2 mm or of substantially 1 mm. In other words, the ball segment, in particular the ball base section and/or the ball support section, may be constructed from of at least one plate-shaped component. Furthermore, the ball segment, in particular the ball base section and/or the ball support section, may be shell- or cup-shaped, and in particular be adapted in is shape to the shape of the foot to be treated.

According to an embodiment, the ball segment can be provided in the form of a clamping bracket. By this configuration, the second corrective force can be provided in the form of a clamping force induced by elastic deformation of the ball segment.

The foot orthosis may be configured such that, in the state of the foot orthosis decoupled from the foot, the ball segment is arranged in a rest position in which the ball segment is not elastically deformed. In the foot orthosis fastened state, however, the ball segment may be arranged in a clamping position in which the ball segment is elastically deformed. The ball segment, in particular the ball support section, may be elastically deflected in is clamping position relative to its rest position in a direction opposite to the second corrective force. For example, in the clamping position, the ball support section, in particular an end section of the ball support section, may be deflected or translationally displaced relative to the rest position by at least 0.1 cm or by at least 0.2 cm or by at least 0.5 cm, in particular along the direction opposite to the second corrective force.

In the clamping position, the ball segment, by its elastic deformation, may be loaded or pre-loaded in the direction of the second corrective force. The clamping force exerted on the ball segment, in particular on the ball support section, may correspond to the second corrective force.

The foot orthosis may further comprise a metatarsal segment, which may be connected to the ball segment in a force-transmitting and a torque-transmitting manner. The metatarsal segment may be configured to fasten the foot orthosis to the foot in a metatarsal region. The metatarsal segment may be provided in the form of a bracket or clamp which, in the fastened state, at least partially engages around a metatarsal side region, in particular a lateral metatarsal region.

The metatarsal segment may be configured and designed to, in the fastened state, exert a holding force on the metatarsal. The holding force may act in a direction parallel to the first corrective force and, together with the first corrective force, provide a counterforce to the second corrective force.

By interaction of these forces, the foot orthosis can be stably held on the foot to be treated in a position for therapeutic treatment. By this embodiment, the foot orthosis can be fastened to the foot to be treated in the way of a clamp or cramp.

In an embodiment, the holding force exerted by the metatarsal segment may have a therapeutic effect on the foot to be treated, contributing in particular to the therapeutic effect of the first and second corrective force and/or providing a further therapeutic effect distinct therefrom. For example, the further corrective force exerted by the metatarsal segment may cause the arch of the foot to straighten up. To support this effect, the foot orthosis may further comprise a foot cushion, also referred to as pad, which is arranged below the sole of the foot in the metatarsal region, in particular below the arch of the foot. Such a foot cushion may be detachably connected to the metatarsal segment and/or the ball segment.

The metatarsal segment, which in particular may be provided in the form of a bracket or clamp, may be configured to, in the fastened state, receive and transmit shearing forces and/or bending forces, in particular in direction of the holding force and/or between the ball segment and the metatarsal to be treated, in order to exert the holding force.

The metatarsal segment provided in the form of a bracket is designed such that, in the state fastened to the foot, the metatarsal segment partially engages around the metatarsal region in the circumferential direction. In other words, in the fastened state, the metatarsal segment extends in the metatarsal circumferential direction along the metatarsus, for example, over a radian of at least ½ π rad about a metatarsal longitudinal axis. Such an arrangement enables that, in the fastened state, the ball segment, in particular the ball support section, is arranged opposite the metatarsal segment, in particular opposite to an end section of the metatarsal segment, to ensure stable holding of the foot orthosis on the foot to be treated.

With regard to the geometric design, the metatarsal segment provided hi the form of a bracket can be plate-shaped. For example, with regard to its geometric design, the metatarsal segment may be belt- or band-shaped any may be bending resistant. Thus, the metatarsal segment may be provided with a contact surface for the metatarsus and an opposite support surface. In the fastened state, the metatarsal may lie against, in particular may directly contact, the contact surface of the metatarsal segment.

To apply or exert the holding force to or onto the metatarsus, the metatarsal segment may comprise a metatarsal support section which, in the fastened state, lies against the metatarsus, in particular against a lateral side thereof. In other words, the metatarsal segment may be configured to apply or exert the holding force to or onto the metatarsus, wherein the holding force points in direction of or coincides with, in particular substantially coincides with, the medial direction. The metatarsal support section may be provide by an end section of the metatarsal segment.

The metatarsal segment may further comprise a metatarsal base section, which may extend along the sole or dorsum of the foot to be treated. In other words, the metatarsal base section may be arranged plantar and/or dorsal. According to one embodiment, the metatarsal base section may have a shape adapted to the shape of the arch of the foot to be treated. The metatarsal base section can be connected to the metatarsal support section in a force-transmitting and/or torque-transmitting manner, for example integrally connected or adhesively bonded.

The metatarsal support section may be provided in the form of a limb or may comprise a limb, and in particular may form a bending spring. In particular, the metatarsal support section can be thin-walled. Furthermore, the metatarsal support section may be arranged substantially vertically.

The metatarsal support section may be arranged laterally to the metatarsus. Thus, the metatarsal support section may directly exert the holding force on the metatarsus by pressing on the metatarsus in the medial direction. Alternatively, the metatarsal support section may comprise or be provided with a support band, in particular in the form of a strap, loop, band, bet, etc. The support band may contact a lateral side of the metatarsus and exert the holding force thereon, wherein the support band may be subjected to a tensile force at its end sections accordingly and can be connected to the metatarsal segment via its end section. For doing so, the support band may be fastened to a limb of the metatarsal support section that is arranged medially to the metatarsus and/or the support band may be connected to the metatarsal base section in a force-transmitting manner. In the fastened state of the foot orthosis, the support band may at least partially be arranged circumferentially around the metatarsal region. In other words, in the fastened state, the metatarsus may be arranged in the support band such that the support band at least partially lies against the metatarsus in the circumferential direction. The support band may be tensile stiff or elastic, in particular along its circumferential direction.

The metatarsal segment, in particular the metatarsal base section and/or the metatarsal support section, may be thin-walled, thereby contributing to a compact design of the foot orthosis. In particular, the metatarsal segment, in particular the metatarsal base section and/or the metatarsal support section, may have a maximum wall thickness of less than 3 mm or less than 2 mm or substantially 1 mm. In other words, the metatarsal segment, in particular the metatarsal base section and/or the metatarsal support section, may be contrasted from at least one plate-shaped or shell-shaped component. Furthermore, the metatarsal segment, in particular the metatarsal base section and/or the metatarsal support section, may be shell- or cup-shaped, and in particular may be adapted in its shape to the shape of the metatarsus to be treated.

According to an embodiment, the metatarsal segment may be provided in the form of a clamping bracket. By this configuration, the holding force can be provided in the form of a clamping force or bending force induced by an elastic deformation of the metatarsal segment.

The foot orthosis may be configured such that, in the state of the foot orthosis decoupled from the foot, the metatarsal segment is arranged in a rest position in which the metatarsal segment is not elastically deformed. In the state of the foot orthosis fastened to the foot, however, the metatarsal segment may be arranged in a clamping position in which the metatarsal segment is elastically deformed. In its clamping position, the metatarsal segment, in particular the metatarsal support section, may be elastically deflected with respect to its rest position in a direction opposite to the direction of the holding force. For example, in the clamping position, the metatarsal support section, in particular an end section of the metatarsal support section, may be deflected or translationally displaced relative to its rest position by at least 0.3 cm, for example by at least 0.5 cm or by at least 1.0 cm, in particular along the direction opposite to the holding force.

In the clamping position, the metatarsal segment, by its elastic deformation, may be loaded or pre-loaded in direction of the holding force. The clamping force exerted on the metatarsal segment, in particular on the metatarsal support section, may correspond to the holding force. Alternatively, the absolute value of the clamping force exerted on the metatarsal segment, in particular on the metatarsal support section, may be substantially equal to an absolute value of the holding force.

The toe segment and/or the ball segment and/or the metatarsal segment may be made of a plastic material, in particular of thermoplastics or a thermoplastic elastomer, etc. The toe segment and/or the ball segment and/or the metatarsus segment may be manufactured by an additive manufacturing process or an injection molding process. Further, the individual segments may comprise different materials, in particular different plastic materials, which can have different material properties. The use of an additive manufacturing process or an injection molding process makes it possible that the individual segments are provided integrally and yet can be made of different materials and can have different material properties. In other words, the sections of the individual segments consisting of different materials can be integrally or adhesively bonded to each other.

According to a further development, different sections of the individual segments may be provided with a different stiffness. For example, the respective support section of the different segments may be provided with a lower stiffness compared to the associated base section. In other words, the toe segment and/or the ball segment and/or the metatarsal segment may comprise the base section and the support section applying the first corrective force or the second corrective force or the holding force to the foot in the fastened state, wherein the support section may have a lower stiffness compared to the base section, in particular in view of shearing and/or bending forces in the direction of the first or second corrective force. In particular, the toe support section may have a lower stiffness compared to the toe base section and/or the ball support section may have a lower stiffness compared to the ball base section and/or the metatarsal support section may have a lower stiffness compared to the metatarsal base section. For this purpose, the structural design of the individual segments may correspondingly be adapted, for example by providing the support section with a lower material thickness compared to the corresponding base section. Alternatively or additionally, different materials may be used within the individual segments. For example, the support section can be made of a material that has a lower modulus of elasticity and/or a lower strength and/or a lower hardness than the material from which the corresponding base section is made of.

According to a further development, the toe support section and the toe base section may be integrally formed. Alternatively or additionally, the ball support section and the ball base section may be integrally formed. Alternatively or additionally, the metatarsal support section and the metatarsal base section may be integrally formed.

Alternatively or additionally, the foot orthosis may comprise a heel segment which may be fastened to the heel or a rear section of the foot to be treated and which may exert, in the fastened state, a holding force on the rear section of the foot. The heel segment may be provided alternatively or additionally to the metatarsal segment and may be configured with corresponding features. In other words, the features described herein in connection with the metatarsal segment may be regarded as being disclosed for the heel segment too.

As described above, the toe segment and the ball segment are pivotably coupled relative to one another by means of a joint unit. By providing the foot orthosis with the joint unit pivotably connecting the toe segment and the ball segment, a toe treated by the foot orthosis can be moved along its flexion-extension movement direction. In other words, the joint unit may be configured such that, in the fastened state, the toe to be treated is movable relative to the metatarsophalangeal joint in the flexion-extension direction. Accordingly, the joint unit may be configured such that the toe segment and the ball segment are pivotable relative to each other about a pivot axis, the pivot axis being parallel or substantially parallel to the first and the second corrective force and/or parallel or substantially parallel to a joint axis of the metatarsophalangeal joint in the direction of flexion and extension. Further, the pivot axis may coincide or substantially coincide with the joint axis.

Furthermore, the joint unit may be configured to transmit shearing and/or bending forces between the toe segment and the ball segment to exert the first and/or the second corrective force on the foot. In other words, the joint unit of the foot orthosis may be configured to, in the fastened state, transmit forces parallel to the corrective forces, in particular shearing or bending forces to generate the first and/or the second corrective force. In other words, the entire foot orthosis is suitable for transmitting along its longitudinal axis internal forces or stresses in the direction of the corrective forces. Accordingly, the joint unit may be provided such that, in the fastened state of the foot orthosis, a relative pivoting movement between the toe segment and the ball segment is locked about an axis arranged orthogonally to the pivot axis.

In this way, the foot orthosis provides a splinted toe with sufficient freedom of movement so that the foot orthosis supports the foot in its natural waking movement and at the same time has a therapeutic effect on it. This allows the foot orthosis to be used in the patient's everyday life, which increases the patient's willingness to wear the foot orthosis and thus the acceptance and success of the therapeutic treatment.

In a further development, the joint unit can be provided by structurally engaged regions, in particular end sections, of the toe segment and the ball segment. In particular, the joint unit may be provided by engaged regions, in particular engaged end sections, of the toe base section and the ball base section. The joint unit may at least partly be provided by the engaged regions of the toe segment and the ball segment, or may entirely be constituted by the engaged regions of the toe segment and the ball segment. The section of the toe segment and/or the ball segment forming the joint unit may be an integral part of the toe segment, in particular the toe base section, and/or the ball segment, in particular the ball base section. In this way, a simple design of the foot orthosis may be ensured by using a small number of components.

According to one embodiment, the joint unit may be arranged laterally at the foot in the fastened state. In particular, the joint unit may be arranged at a medial side of the foot hi the fastened state. In this configuration, the joint axis of the joint unit, which coincides with the pivot axis, may coincide or substantially coincide with the joint axis of the metatarsophalangeal joint. According to this embodiment, the section of the toe segment forming the joint unit and/or the correspondingly designed further section of the ball segment further forming the joint unit may be arranged in the region of the metatarsophalangeal joint to be treated and may exert the second corrective force on the metatarsophalangeal joint.

Alternatively or additionally, in the fastened state, the joint unit may be arranged in the region of the sole of the foot and/or the dorsum of the foot. Accordingly, the joint unit may extend along the sole of the foot and/or along the dorsum of the foot, in particular along a direction parallel or substantially parallel to the first and the second corrective force.

In a further development, the joint unit may comprise at least one swivel joint and/or one bending joint. In the context of the present disclosure, a swivel joint refers to a joint unit in which two relatively pivotable and engaged sections of the joint are pivotably or rotatably mounted relative to one another. In contrast, a flexural joint describes a joint unit in which two engaged components of the joint can be pivoted relative to each other by elastic or plastic deformation.

The swivel joint may be provided in the form of a ring joint, in which a contact surface or a sliding surface between sections of the swivel joint, which are pivotable relative to one another, is arranged annularly around the joint or pivot axis of the swivel joint. By this configuration, the joint unit may be particularly robust against bending forces and bending torques, thereby allowing the joint unit to transmit and induce high forces and torques into the foot to be treated, while at the same time providing a compact design of the foot orthosis.

In a further development, the swivel joint, in particular when being provided in the form of a ring joint, may be provided in the form of a hollow trunnion swivel joint. In the context of the present disclosure, the term “hollow trunnion swivel joint”, which may also be referred to as a hubless swivel joint, refers to a joint unit which, along its pivot axis, is at least partially hollow to provide a recess. In other words, the components forming the swivel joint are provided hollow along the pivot axis such that the swivel joint is provided with the recess or a through hole around and along its pivot axis.

The swivel joint may comprise components which are pivotable relative to each other about the pivot axis and which are guided relative to each other in the swivel joint. The region configured for guiding the components, which may be formed by bearing points and/or bearing surfaces, in particular by contact surfaces and/or sliding surfaces, can be arranged circumferentially around the pivot axis and spaced apart therefrom.

The region configured for guiding the components of the swivel joint may be arranged spaced apart from the swivel axis by a guiding radius. In particular, the guiding radius may indicate an average radius of the region configured for guiding the components of the swivel joint about the pivot axis. The guiding radius may substantially lie within the range of an outer radius of the swivel joint, which describes an extension of the swivel joint in the radial direction. The guiding radius may be at least 70% of the outer radius of the swivel joint. In particular, the guiding radius may be at least 80% or at least 90% of the outer radius.

The swivel joint may be provided such that, in the fastened state of the foot orthosis, the swivel joint is arranged on the foot and the metatarsophalangeal joint in such a way that the swivel joint is arranged circumferentially around the sideways protruding section of the metatarsophalangeal joint, for example around the pseudoexostosis, wherein the sideways protruding section of the metatarsophalangeal joint is at least partially received in the recess of the swivel joint.

The recess may be provided in the form of a recess that is open on at least one side. In the fastened state, the recess can be open in the direction of the foot. Alternatively, the recess may be provided in the form of a through hole, in particular in the form of a through hole extending along the pivot axis. In other words, the recess may extend along the entire width or thickness of the swivel joint. In the context of the present disclosure, the terms “width” and “thickness” of the swivel joint refer to an extension of the swivel joint along the pivot axis. The swivel joint may have a width, in particular maximum width, of 1.0 cm or 0.6 cm.

The foot orthosis may be configured such that, in the fastened state, the sideways protruding section of the metatarsophalangeal joint extends along at least 50% or at least 70% or at least 80% of the maximum width of the swivel joint. Furthermore, the foot orthosis may be configured such that, in the fastened state, the sideways protruding section of the metatarsophalangeal joint protrudes or substantially protrudes through the recess, in particular through the through hole, along the pivot axis. In this way, the foot orthosis can be kept particularly close to the foot.

The swivel joint may comprise a side wall which confines the recess and which is arranged circumferentially around the pivot axis. The side wall may have a minimum radius of curvature of 1 mm or 2 mm or 5 mm. In other words, the swivel joint may be designed such that the side wall at no point has a radius of curvature that is less than the minimum radius of curvature. The reciprocal value of the radius of curvature corresponds to the curvature of the side wall, in particular to a curvature of an inner surface of the side wall facing the recess. In this way, t may be ensured that the region of the swivel joint adjacent to the sideways protruding section of the metatarsophalangeal joint is not provided with sharp edges in order to prevent pressure peaks on the patient's foot when wearing the foot orthosis.

The recess may have a minimum diameter, in particular along a direction transverse to the pivot axis or around the pivot axis, of at least 1.5 cm or at least 2.0 cm or at least 2.5 cm. For example, the recess may have a circular or elliptical shape in cross-section along the pivot axis with a minimum diameter of at least 1.5 cm or at least 2.0 cm or at least 2.5 cm. For example, the diameter may be 3.0 cm or substantially 3.0 cm.

The shape of the recess, in particular its cross-sectional shape and diameter, may be adapted to the foot to be treated, in particular to the shape of the sideways protruding section of the metatarsophalangeal joint. This may be done on the basis of orthopedic or physiological classification specific to the user group. For example, foot orthoses can be provided in this way for user groups with feet of different sizes and/or user groups with sideways protruding sections of the metatarsophalangeal joint, in particular with pseudoexostosis, of different sizes.

The pivot axis of the swivel joint may coincide with, i.e. may be aligned with, or may be substantially coincide with the base joint axis of the metatarsophalangeal joint, in particular with the flexion-extension movement axis. Alternatively or additionally, the pivot axis of the pivot joint may be arranged parallel or substantially parallel to the first and/or the second corrective force.

The swivel joint may be designed such that, in the fastened state of the foot orthosis, relative pivotal movement between the toe segment and the ball segment is locked about an axis arranged obliquely or orthogonally to the pivot axis. The swivel joint may be designed such that relative pivotal movement is permitted only about the pivot axis. In other words, the swivel joint may be structurally provided such that pivotal movement about the pivot axis is released, while pivotal movement about an axis oblique or perpendicular to the pivot axis is blocked. In this way, a simple and compact design of the swivel joint may be provided.

The joint unit, in particular the swivel joint, may comprise a first joint element coupled to the toe segment, in particular integrally connected or adhesively bonded thereto, and a correspondingly designed second joint element which is engaged with the first joint element and which is coupled to the ball segment, in particular integrally connected or adhesively bonded thereto. The first joint element and the second joint element may be form-fittingly engaged along the pivot axis, in particular in a first direction and a second opposite direction along the pivot axis, and/or transversely to the pivot axis of the swivel joint.

The swivel joint may be designed such that, in the fastened state, the second joint element is arranged between the foot and the first joint element. In this way, it may be prevented that during a flexing movement of the splinted toe, the section of the swivel joint lying against the foot is pivoted relative to the sideways protruding section of the metatarsophalangeal joint. This may increase wearing comfort of the foot orthosis.

The second joint element may form a joint pin of the swivel joint which guides movement around the pivot axis of a joint ring formed by the first joint element. Alternatively, the first joint element may form the joint pin and the second joint element may form the joint ring. The joint pin is can be provided in the form of a hollow pin, the hollow section of which constitutes the recess. The joint ring and the joint pin may be designed and engaged in such a way that they are form-fittingly engaged along the pivot axis, in particular in the first direction and the opposite second direction along the pivot axis.

The joint pin, as regards is geometric design, may be adapted to the shape of the joint ring. The joint ring may comprise a first guiding surface which may be formed correspondingly to a second guiding surface of the joint pin. The first and the second guiding surface, which in particular constitute sliding and bearing surfaces, may be engaged with one another, in particular substantially without clearance or with a predetermined clearance. During pivoting movement, the first and the second guiding surfaces can be moved relative to each other.

The first guiding surface of the joint ring may be or comprise a surface which is oriented radially inward, i.e. which faces the pivot axis. The first guiding surface may extend circumferentially around the pivot axis and may be arranged annularly about the pivot axis.

The second guiding surface of the joint pin may be or comprise a surface oriented radially outward which in particular may constitute a lateral surface of the joint pin. The second guiding surface may extend circumferentially around the pivot axis and may be arranged annularly about the pivot axis.

Further, the joint pin may comprise a circumferential radial shoulder at a distal and/or proximal end, wherein the shoulder, in particular by way of a snap hook, provides a form-fitting connection or undercut securing between the joint ring and joint pin in direction of the pivot axis. In order to provide a substantially flat outer contour or outer surface on the medial outer side of the foot orthosis, the radial shoulder may be received in a correspondingly designed receptacle or recess on the joint ring. Alternatively or additionally, a separate locking ring may be provided which can be inserted into a correspondingly designed groove at the joint ring or joint pin. The radial shoulder may extend along the pivot axis in such a way that t overlaps the joint ring in the axial direction of the swivel joint and in particular engages around the joint ring. By this arrangement, the foot to be treated can be prevented from coming into contact with the receiving groove or the joint ring, thereby increasing comfort for a patient.

The first and the second guiding surface may comprise at least one axially delimiting side surface, for example two opposing side surfaces, to provide the form-fitting connection along the pivot axis.

By this configuration, forces, in particular bending forces, in direction of the pivot axis of the swivel joint can be transmitted between the components.

According to one configuration, the joint pin, in particular the second joint element, or the joint ring may be provided with a receiving groove which is arranged circumferentially around the pivot axis and which extends in radial direction. The receiving groove may be delimited, in particular laterally delimited, in the axial direction along the pivot axis. The inner surfaces of the receiving groove may constitute a contact and sliding surface, i.e. the first or the second guiding surface. In other words, the receiving groove may form a substantially U-shaped contact or sliding surface in longitudinal section along the pivot axis. The correspondingly designed guiding surface may be provided at a connecting ring correspondingly designed to the receiving groove. The connecting ring may be guided in the receiving groove and may be rotated relative to the receiving groove in the circumferential direction about the pivot axis. In the engaged state of the receiving groove and the connecting ring, the connecting ring is arranged in the receiving groove such that the first joint element and the second joint element are form-fittingly connected to one another in axial direction of the pivot axis.

In other words, the joint pin may be provided with a receiving groove in which a correspondingly designed connecting ring of the joint ring is guided. Alternatively, the joint ring may be provided with the receiving groove in which the correspondingly designed connecting ring of the joint pin is guided. The joint unit may be designed such that, in the fastened state of the foot orthosis, the second joint element is arranged between the foot and the first joint element, wherein a connecting stud of the second joint element, which forms the receiving groove, circumferentially delimits, in particular radially delimits, the recess or through hole of the swivel joint. The connecting stud may extend along the pivot axis such that the connecting stud overlaps the first joint element, in particular the connecting ring, in axial direction of the joint unit and in particular engages around or encompasses it. By this configuration, it can be prevented that the foot to be treated comes into contact with the receiving groove or the connecting ring, thereby increasing wearing comfort for a patient.

According to a further development, the joint unit, which ensures freedom of movement, may be arranged along the dorsum of the foot and/or along the sole of the foot in the fastened state. In other words, the joint unit may be arranged plantar, i.e. on the plantar side of the foot or in the region of the sole of the foot, and/or dorsal, i.e. on the dorsal side of the foot or in the region of the dorsum of the foot. This enables a structurally simple and space-saving design of the foot orthosis.

More specifically, the joint unit, which may be arranged dorsal and/or plantar, may extend along a foot width direction or parallel to the first and the second corrective force, in particular along the entire width of the toe base segment and/or the ball base segment. By such a configuration, thin and flat shapes of the toe base segment and/or the ball base segment can be achieved. Thus the toe base segment and/or the ball base segment may have a high geometrical moment of inertia against stresses exerted in direction of the corrective forces, while at the same time being provided with small cross-section surfaces, thereby contributing in particular to a compact design of the foot orthosis.

The joint unit, which may be arranged dorsal and/or plantar, may be provided in the form of a bending joint or a flexure joint, in particular in the form of a single-joint hinge, for example in the form of a film hinge. The film hinge may be provided with reinforced ends such that is robustness against transverse forces, in particular shearing forces, is increased. Further, the connecting section may comprise a plurality of axe-parallel or substantially axe-parallel single-joint hinges, for example in the form of a plurality of film hinges which are arranged in series. The plurality of film hinges arranged in series may, at their hinge ends, be connected to one another by suitable means and may be reinforced with respect to transverse forces. In this way, the pivoting mobility of the foot orthosis may be further increased and the wearing comfort improved.

Alternatively or additionally, the joint unit, which may be arranged dorsal and/or plantar, may comprise a bending joint. The foot orthosis may be designed such that, compared to regions of the toe segment and the ball segment which are arranged adjacent to the bending joint, the bending joint provides less bending stiffness to pivoting movements about the pivot axis.

For this purpose, the bending joint may be provided with a geometrical design which reduces its bending stiffness, in particular its geometrical moment of inertia, with respect to bending stresses about the pivot axis. For example, the foot orthosis in the region of the bending joint may have a smaller thickness compared to regions of the toe segment and ball segment adjacent thereto.

Alternatively or additionally, the bending joint may have a material composition that reduces its bending stiffness. For example, the foot orthosis may be designed such that it is made of a material in the region of the bending joint that has a lower modulus of elasticity and/or a lower strength and/or a lower hardness compared to the material of regions arranged adjacent thereto.

Alternatively or additionally, the foot orthosis may be made of a composite material in the region of the joint unit that has fibers, in particular tensile stiff fibers that extend transversely to the pivot axis or parallel to the longitudinal axis of the foot orthosis. By this configuration, a load-oriented and durable design of the foot orthosis may be ensured.

According to an embodiment, the ball segment, in particular the ball base section, may be coupled to the metatarsal segment, in particular to the metatarsal base section, in a force-transmitting and/or torque-transmitting manner. According to one embodiment, the ball segment, in particular the ball base section, and the metatarsal segment, in particular the metatarsal base section, may be coupled to one another such that the ball segment and the metatarsal segment are translationally displaceable relative to each other, in particular along a longitudinal axis and/or a transverse axis of the foot orthosis. Further, the ball segment and the metatarsal segment may be designed such that they can be fixed in a desired position relative to one another, in particular can be fixed in a force-fitting and/or form-fitting manner. By this configuration, the foot orthosis can be adapted to the size of a foot to be treated. For doing so, the coupling between the ball segment and the metatarsal segment can be selectively locked or released. In a released state of the coupling between the ball segment and the metatarsal segment, the ball segment and the metatarsal segment can be translationally displaced relative to each other to adjust a desired relative position. Thereupon, the coupling can be locked by force and/or form closure such that relative translational movement between the ball segment and the metatarsal segment is locked.

Alternatively or additionally, the ball segment may be coupled to the metatarsal segment via a further joint unit, which may be provided in a manner corresponding to the dorsal and/or plantar joint unit as described above.

In a further development, the foot orthosis, in particular the toe segment and/or the ball segment and/or the metatarsal segment, on its/their inside, may be provided with a support layer or coating, in particular a cushioning coating, for example a cushioning polyvinyl chloride (PVC) coating or a polyurethane (PU) coating, wherein in particular the coating is softer, i.e. has a lower hardness, compared to the section of the foot orthosis carrying the coating. The coating can be applied to the inside of at least one of the sections or segments of the foot orthosis. The term “inside” refers to those regions of the foot orthosis that face the foot in the fastened state. The cushioning coating may be applied in an additive process in the form of a solid or gel to the inside surfaces of the foot orthosis or the limbs. For example, the cushioning coating may be applied by lamination or laminating. Further, a suitable PVC material or PU material may be melted and pressed onto the splint to create the coating. Additionally or alternatively, the coating may be applied in a liquid state, in particular by dipping, doctoring, roller application, spraying, foaming or any other suitable method.

### BRIEF DESCRIPTION OF THE FIGURES

Further embodiments of the disclosure are explained in more detail below with reference to the figures, which schematically show in:

FIG. 1 a perspective view of a foot orthosis according to one embodiment in a state fastened to a patient's foot;

FIGS. 2 and 3 the foot orthosis depicted in FIG. 1, wherein for overview reasons the foot is not shown;

FIG. 4 to 9 the foot orthosis depicted in FIGS. 1 and 2 in a state decoupled from the foot;

FIG. 10 a perspective view of the foot orthosis according to a further embodiment;

FIG. 11 a perspective view of the foot orthosis according to a further embodiment;

FIG. 12 a perspective view of the foot orthosis according to a further embodiment; and

FIGS. 13 and 14 the foot orthosis according to a further embodiment.

### DETAILED DESCRIPTION

In the following, embodiments are described on the basis of the Figures. In the Figures, identical, similar or similarly acting elements are denoted by identical reference numerals and a repeated description thereof may be omitted in order to avoid redundancies.

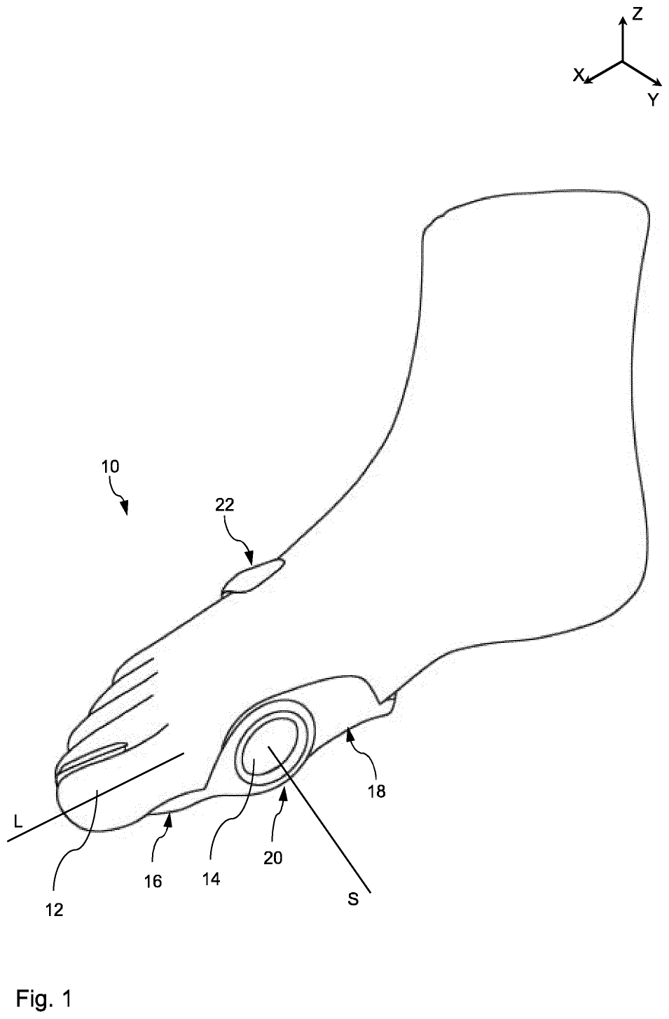

FIG. 1 shows an embodiment of a foot orthosis 10 for correcting foot malpositions. In particular, the foot orthosis 10 shown in FIG. 1 is intended and provided for treating hallux valgus, i.e. the pathological malposition of a big toe 12, also referred to as valgus position of the big toe, and of a big toe metatarsophalangeal joint 14, also referred to as varus position of the big toe metatarsophalangeal joint. Further, the shown foot orthosis 10 can be used for preventing hallux valgus. In the following, for simplicity reasons, the term “big toe” will be abbreviated by the term “toe” and the term “metatarsophalangeal joint of the big toe” will be abbreviated by the term “metatarsophalangeal joint of the toe”.

As can be gathered from FIG. 1, the foot orthosis 10 is configured to be fastened to a foot to be treated in the manner of a clamp, in particular a tension clamp, and to be fixed thereto in a predetermined position. The foot orthosis 10 is designed and configured to, in the shown fastened state, therapeutically act on the foot by selectively applying corrective forces to the foot, in particular to a region of the toe 12 and the metatarsophalangeal joint 14, as will be further specified below.

The shown foot orthosis 10 is designed to be used on the patient's right foot. For treatment of the patient's left foot, a foot orthosis may be used that is mirror symmetric to the configuration shown in FIG. 1.

The foot orthosis 10 comprises a toe segment 16 configured to be fastened to the toe 12 and a ball segment 18 configured to be arranged in the region of the metatarsophalangeal joint 14 and configured to be fastened to the metatarsophalangeal joint 14. The toe segment 16 and the ball segment 18 are pivotably connected relative to one another by means of a joint unit 20. The foot orthosis 10 further comprises a metatarsal segment 22 configured to be fastened to or in the region of the metatarsus of the foot. The metatarsal segment 22 is coupled to the ball segment 18 in a force- and torque-transmitting manner.

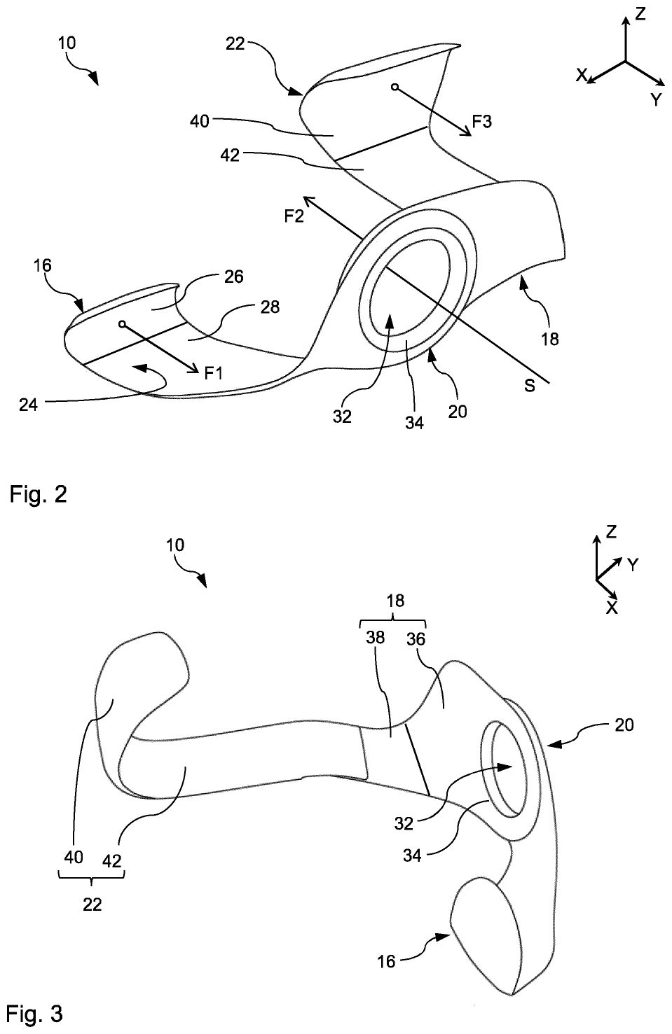

In the state fastened to the foot, as indicated in FIG. 2, the foot orthosis 10 is configured to exert a first corrective force F1 on the toe 12 via the toe segment 16, to exert a second corrective force F2 on the metatarsophalangeal joint 14 via the ball segment 18, and to exert a holding force F3 via the metatarsal segment 22 to the metatarsus. The second corrective force F2 is opposed to the first corrective force F1, in particular is parallel to and spaced apart from the first corrective force F1. The holding force F3, which may be a third corrective force, points in direction of the first corrective force F1 and is parallel to and spaced apart from the first corrective force F1.

The forces F1, F2, F3, acting on the treated foot during use of the foot orthosis 10, are illustrated in FIG. 2, in which the foot is not shown for reason of better visualization. For simplicity reasons, the corresponding forces F1, F2, F3 are shown in the form of individual vectors, wherein each of the forces, of course, is distributively exerted via a corresponding contact surface between the foot and the foot orthosis during use.

As will be specified below, the individual segments 16, 18, 22 of the foot orthosis 10 are elastically deformable, wherein the different forces F1, F2, F3 exerted on the foot by the foot orthosis 10 are provided in the form of clamping and/or bending forces induced by elastic deformation of the foot orthosis 10. By this configuration, it may be ensured that the forces F1, F2, F3 exerted by the foot orthosis 10 are persistently applied to the foot even when the foot is moving and is shape changes during use.

The first corrective force F1, the second corrective force F2, and the holding force F3 are arranged parallel or substantially parallel and are spaced apart relative to each other. The first corrective force F1 and the holding force F3 point in the medial direction and are parallel or substantially parallel to a transverse axis Y of the foot orthosis 10. The second corrective force F2 points in the lateral direction. The first corrective force F1, the second corrective force F2, and the holding force F3 are further arranged orthogonal or substantially orthogonal to a longitudinal axis X and a vertical axis Z of the foot orthosis 10.

The toe segment 16 is provided in the form of a bracket or clamp, in particular in the form of a tensioning or bending clamp, which engages around, i.e. encompasses, the toe 12 in the fastened state. In the shown configuration, the toe segment 16 extends from a medial side of the foot, starting from the ball segment 18, along a lower side of the toe 12 to a lateral side of the toe 12. Thus, the toe segment 16 extends in sections, i.e. partially, along the lower side of the toe 12. Alternatively, the toe segment 16 may extend along an upper side of the toe 12. In the shown configuration, the toe segment 16 extends along the toe over a radian of one π rad around the longitudinal axis L such that the toe segment 16 extends from one side of the toe 12 and the metatarsophalangeal joint 14 to the opposite side of the toe 12, as shown in FIG. 1.

The toe segment 16 comprises a contact surface 24 for the toe 12. The contact surface 24 is provided in the form of a turning surface, the orientation of which, i.e. the surface normal of which, changes along the longitudinal toe axis L and can point to the longitudinal toe axis. By this configuration, the contact surface 24 and thus also the toe segment 16 extend along a helical line around the toe 12.

The toe segment 16 is configured to, in the fastened state, transmit shearing forces and/or bending forces in direction of the first corrective force F1 between the joint unit 20 and the toe 12 to be treated, thereby contributing in generating the first corrective force F1.

In order to exert the first corrective force F1 on the toe 12, the toe segment 16 comprises a toe support section 26 which, in the fastened state of the foot orthosis 10, contacts a lateral side of the toe 12, i.e. a side of the toe 12 pointing in lateral direction. The toe support section 26 is formed by a distal end section of the toe segment 16. The toe segment 16 further comprises a toe base section 28 integrally coupled and adhesively bonded to the toe support section 26 and arranged adjacent thereto, as indicated by a dotted line in FIG. 2.

The toe segment 16, in particular the toe support section 26, is provided in the form of a bending spring or spring element. Thus, the first corrective force F1 is provided in the form of a clamping force or bending force induced by an elastic deformation of the toe segment 16. In other words, the toe segment is configured such that, in the fastened state, the toe segment 16 is arranged in a clamping position in which the toe segment 16 is elastically deflected in a direction opposite to the first corrective force F1 with respect to a rest position of the toe segment 16 in which the toe segment 16 is arranged in a state of the foot orthosis 10 being decoupled from the foot.

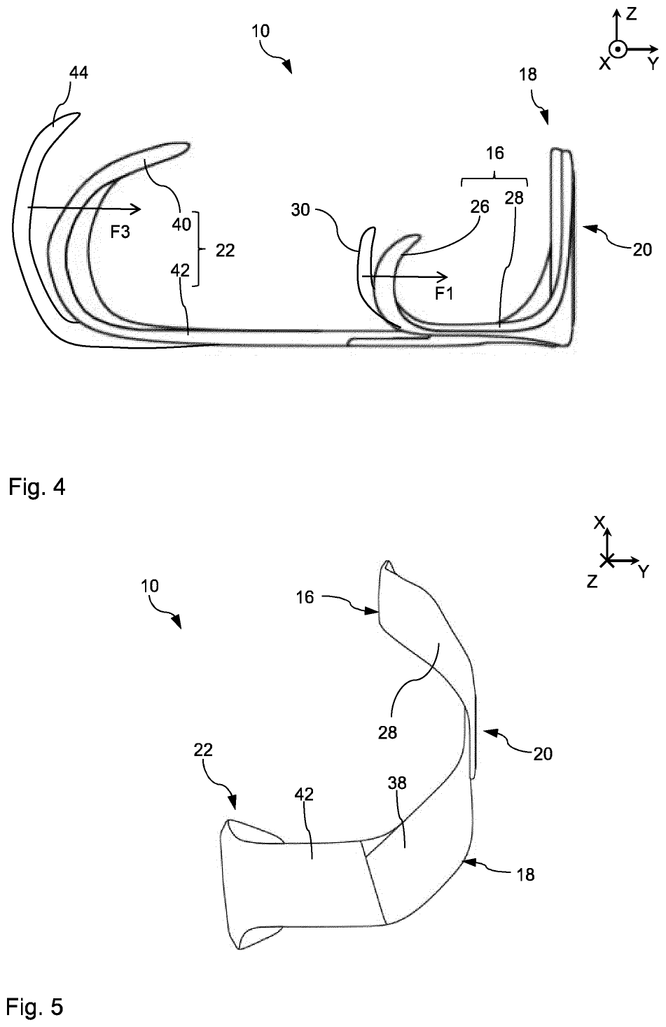

For illustrating this structural configuration of the toe segment 16, FIG. 4 shows the foot orthosis 10 in a state decoupled and disengaged from the foot, in which the toe segment 16 is arranged in its rest position. Further, by a dashed line 30, a state of the toe segment 16 is indicated in which t is arranged in the clamping position, i.e. in the fastened state. In the clamping position, an end section of the toe support section 26 is deflected and translationally displaced along the direction opposite to the first corrective force F1 by at least 0.3 cm or 0.5 cm, for example by at least 1.0 cm, with respect to the rest position.

In the fastened state of the foot orthosis 10, the ball segment 18 is arranged in the region of the metatarsophalangeal joint 14 and lies against the foot in the region of the metatarsophalangeal joint 14. In other words, the ball segment 18 is configured to be engaged with the foot in the region of the metatarsophalangeal joint 14 to provide a force-transmitting coupling between the foot in the region of the metatarsophalangeal joint 14 and the ball segment 18 in the fastened state of the foot orthosis 10. In particular, in the fastened state, the ball segment 18 contacts the foot in a region of a lateral ball-shaped protrusion of the foot, referred to as a pseudoexostosis. In the shown configuration, the ball segment 18 comprises a through hole 32 extending along a pivot axis S of the joint unit 20. An edge 34, in particular a rounded edge 34, of the ball segment 18 radially delimiting the through hole 32 contacts the foot circumferentially around the ball-shaped protrusion. In other words, a section of the metatarsophalangeal joint 14, in particular the ball-shaped protrusion, is received and arranged within the through hole 32. The through hole 32 of the ball segment 18 may have a diameter, in particular a minimum diameter, of at least 2 cm or 2.5 cm along its cross-section.

In order to exert the second corrective force F2 on the foot, the ball segment 18 comprises a ball support section 36 which contacts the ball of the metatarsophalangeal joint in the fastened state of the foot orthosis 10, more specifically which contacts the ball of the metatarsophalangeal joint at a medial and plantar section. The ball segment 18 may further comprise a ball base section 38, which constitutes a section of the ball segment 18 extending along the plantar surface of the foot. The ball base section 38 is integrally coupled and adhesively bonded to the ball support section 36 and arranged adjacent thereto, as indicated by a dotted line in FIG. 3.

The ball segment 18, in particular the ball support section 36, is provided in the form of a bending spring. Thus, the second corrective force F2 is provided in the form of a clamping force or bending force induced by an elastic deformation of the ball segment 18. In other words, the ball segment 18 is configured such that, in the fastened state, the ball segment 18 is arranged in a clamping position in which the ball segment is elastically deflected in a direction opposite to the second corrective force F2 with respect to a rest position in which the ball segment is arranged in a state of the foot orthosis 10 decoupled from the foot. An end section of the ball support section 36 in the clamping position can be deflected and translationally displaced by at least 0.3 cm or 0.5 cm relative to the rest position along the direction opposite to the second corrective force F2. The relative deflection of the ball segment 18 may be less than the relative deflection of the toe segment 16.

The ball segment 18 is connected to the metatarsal segment 22 in a force- and torque-transmitting manner. The metatarsal segment 22 is configured to be fastened to the metatarsus of the foot to be treated. Further, the metatarsal segment 22 is partially arranged opposite to the ball segment 18, in particular opposite to the ball support section 36. The metatarsal segment 22 is provided in the form of a bracket or clamp, in particular a clamping bracket, which in the fastened state of the foot orthosis 10 partially engages around a lateral metatarsal region, in particular a lateral midfoot region. The metatarsal segment 22, provided in the form of a bracket, may be configured to, in the fastened state, receive shearing forces and/or bending forces, in particular in the direction of the holding force, and to transmit these forces between the ball segment 18 and the metatarsus of the foot to be treated.

For applying the holding force to the metatarsus, the metatarsal segment 22 comprises a metatarsal support section 40 which lies against a lateral side of the metatarsus in the fastened state of the foot orthosis 10. The metatarsal support section 40 may be formed by an end section of the metatarsal segment 22. The metatarsal segment 22 further comprises a metatarsal base section 42. The metatarsal base section 42 is integrally coupled and adhesively bonded to the metatarsal support section 40 and is arranged adjacent thereto, as indicated by a dotted line in FIG. 2.

The metatarsal segment 22, in particular the metatarsal support section 40 and the metatarsal base section 42, is provided in the form of a bending spring or spring element. Thus, the holding force F3 is provided in the form of a clamping force or bending force induced by an elastic deformation of the metatarsal segment 22. In other words, the metatarsal segment 22 is configured such that, in the fastened state, the metatarsal segment 22 is arranged in a clamping position in which the metatarsal segment 22 is elastically deflected in a direction opposite to the holding force F3 with respect to a rest position of the metatarsal segment 22 in which the metatarsal segment 22 is arranged in a state of the foot orthosis 10 being decoupled from the foot. This structural configuration is illustrated in FIG. 4 by a further dashed line 44 indicating a state of the metatarsal segment 22 in which the metatarsal segment 22 is arranged in the clamping position, i.e. in the fastened state. An end section of the metatarsal support section 40 in the clamping position, with respect to its rest position, is deflected and translationally displaced along the direction opposite to the holding force F3 by at least 0.5 cm or 1.0 cm, for example 2 cm.

For fastening the foot orthosis to the foot, the metatarsal segment 22 and/or the toe segment 16 optionally may be provided with bandages. For example, the toe segment 16 may use a bandage or a support band to force- and/or from-fittingly fix the toe 12 clamped in the toe segment 16 relative to the toe segment 16. For doing so, the bandage or the support band may lie against the toe 12 at least partially in its circumferential direction and may be connected to the toe segment 16.

Furthermore, the metatarsal segment 22 may be provided with a second bandage or support band, which extends around the metatarsus in its circumferential direction and which is coupled to the metatarsal segment 22 at its ends in order to fix the metatarsus in the metatarsal segment 22 in a form-fitting and/or force-fitting manner.

The toe segment 16 and/or the ball segment 18 and/or the metatarsal segment 22 are thin-walled. In particular, the toe segment 16 and/or the ball segment 18 and/or the metatarsal segment 22 may be formed by or consist of plate-shaped and/or shell-shaped elements having a maximum thickness of less than 3 mm or 2 mm or 1 mm.

The toe segment 16, the ball segment 18 and the metatarsal segment 22 can be made of a plastic material. In particular, a plastic material may be used which is plastically deformable when being subjected to high forces, i.e. which are higher than the corrective forces F1, F2 and the holding force F3, or heat. By this configuration, the foot orthosis may be easily adapted in its geometric design to a foot to be treated. In this way, also the corrective and holding forces F1, F2, F3 induced by elastic deformation may be adapted.

According to a further development, at least one of the support sections 26, 36, 40 may have a lower stiffness, in particular against shearing and/or bending forces in direction of the first or second corrective force F1, F2, compared to the base section 28, 38, 42 arranged adjacent thereto.

Specifically, the at least one support section 26, 36, 40 may be made of a material which has a lower modulus of elasticity or a lower hardness, for example a lower Shore hardness, compared to the material of the adjacent base element 28, 38, 42.

As set forth above, the toe segment 16 and the ball segment 18 are pivotally coupled relative to each other about the pivot axis S by means of the joint unit 20. In the shown configuration, the pivot axis S of the joint unit 20 is arranged in alignment or substantially in alignment with the flexion-extension joint axis of the metatarsophalangeal joint 14. The flexion-extension joint axis is understood to be the joint axis about which the toe 12 is pivoted relative to the metatarsus during flexion and extension movements. This is achieved by arranging the joint unit 20 on the medial side of the foot in the fastened state.

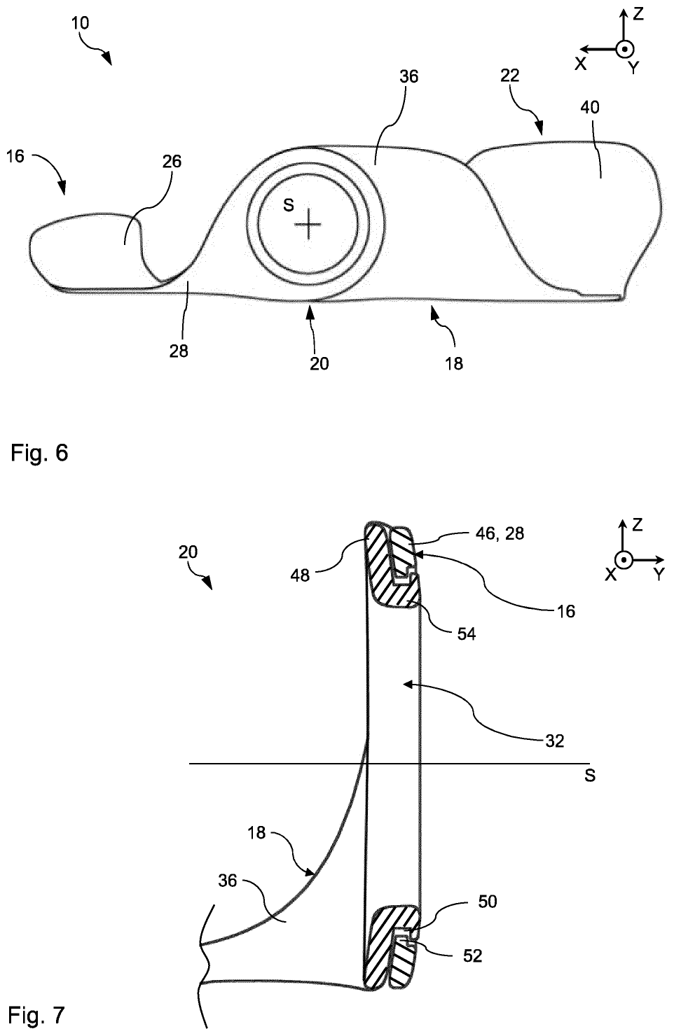

In the configuration shown in FIGS. 1 to 9, the joint unit 20 is provided in the form of a swivel joint, more specifically in the form of a hubless ring joint arranged around the through hole 32. In other words, the joint unit 20 is provided with the through hole 32 or alternatively with a recess, in which the medially protruding section of the metatarsophalangeal joint 14, in particular the pseudoexostosis, is partially received in the fastened state, as previously described in connection with the through hole 32.

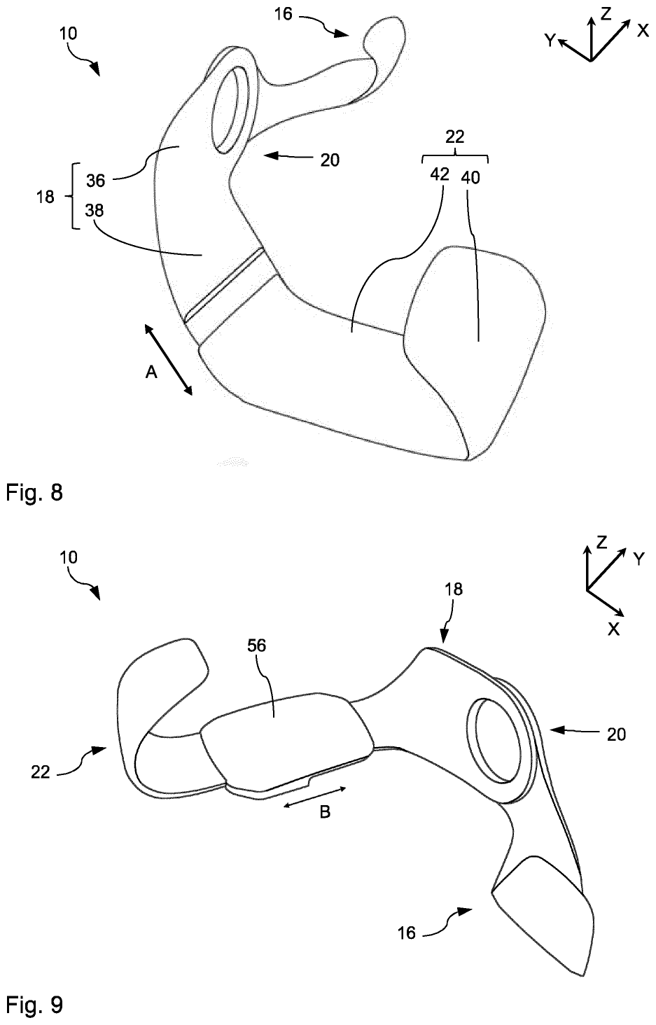

FIG. 7 shows a longitudinal section of the joint unit 20 along the pivot axis S. The joint unit 20 is constituted by at least two components, but is not limited thereto and in alterative embodiments may be constituted by more than two components.

In particular, the joint unit 20 comprises a first joint element 46 coupled to the toe segment 16, in particular integrally coupled or adhesively bonded thereto, and a correspondingly designed second joint element 48 which is engaged with the first joint element 46 and which is coupled to the ball segment 18, in particular integrally connected or adhesively bonded thereto. In particular, the first joint element 46 may be formed by an end section of the toe base section 28 and the second joint element 48 may be formed by an end section of the ball support section 36. The second joint element 48 is provided with a receiving groove 50 which extends circumferentially around the pivot axis S and is engaged with a complementary designed connecting ring 52 of the first joint element 46. The receiving groove 50 further extends in radial direction relative to the pivot axis S such that the receiving groove 50 is delimited in both axial directions along the pivot axis S and comprises an opening in radial outward direction. Via the opening, the connecting ring 52 protrudes into the receiving groove 50. In other words, in longitudinal section along the pivot axis S, the receiving groove 50 has a substantially U-shaped contact or sliding surface for the connecting ring 52. Thus, the receiving groove 50 and the connecting ring 52 are form-fittingly engaged in both directions along the pivot axis S. By this configuration, the connection between the first and the second joint element 46, 48 can be effectively prevented from being unintentionally released and, at the same time, a simple and robust design of the joint unit 20 may be ensured.

The joint unit 20 is designed and configured such that, in the fastened state of the foot orthosis 10, the second joint element 48 is arranged between the foot and the first joint element 46, wherein a connecting stud 54 of the second joint element 48, which forms the receiving groove 50, circumferentially delimits the through hole 32 in radial direction. The connecting stud 54 extends along the pivot axis S such that the connecting stud 54 overlaps the first joint element 46 in axial direction of the joint unit 20, i.e. along the pivot axis S. Specifically, the connecting stud 54 engages around the connecting ring 52.

As shown in FIG. 8, the ball base section 38 and the metatarsal base section 42 are coupled to each other such that they are translationally displaceable relative to each other along an axis that is in a plane with, but transverse to the longitudinal axis X and a transverse axis Y of the foot orthosis 10, as indicated by arrow A in FIG. 8. In other words, the two sections 38, 42 are translationally displaceable relative to each other along the longitudinal axis X and the transverse axis Y of the foot orthosis 10. The foot orthosis 10 is configured such that the two sections 38, 42 can be fixed in a desired position relative to each other in a force-fitting and/or form-fitting manner.

Optionally, the foot orthosis 10 can further comprise a foot cushion 56 which is configured to be releasably coupled to the ball base section 38 and/or the metatarsal base section 42 and is displaceable relative thereto in order to arrange the foot cushion 56 in a patient-specific manner, as indicated by arrow B in FIG. 9.

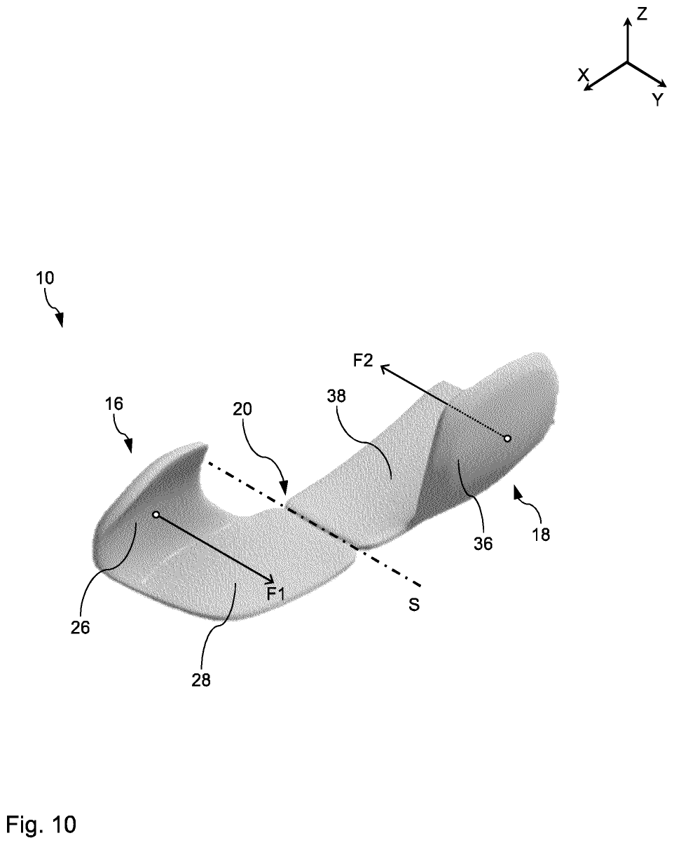

FIG. 10 shows a second embodiment of the foot orthosis 10 that differs from the embodiment shown in FIGS. 1 to 9 in particular in the configuration and arrangement of the toe segment 16, the ball segment 18, and the joint unit 20.

In the embodiment shown in FIG. 10, the joint unit 20 is arranged along the sole of the foot in the state fastened to the foot. In other words, during use, the joint unit 20 is arranged plantar, i.e. on the plantar side of the foot or in the region of the sole of the foot. The joint unit 20 is provided in the form of a bending joint formed by end sections of the plate- or shell-shaped toe base section 28 and the ball base section 38. The joint unit 20 can extend along the transverse axis Y over a length of at least 1 cm. In the shown embodiment, the joint unit 20 extends along the transverse axis Y substantially over the entire width of the engaged end sections of the toe base section 28 and the ball base section 38.

The toe base section 28 and the ball base section 38 extend in a plate- or shell-like manner along a plane parallel to the corrective forces F1, F2. Along this plane, also the joint unit 20 extends and is arranged therein. By this configuration, the foot orthosis 10, in particular the joint unit 20, may be particularly robust against bending and shearing forces in the direction of the corrective forces F1, F2.

The joint unit 20, which is provided in the form of a bending joint, has a lower bending stiffness with respect to pivoting movements about the pivot axis S compared to regions of the toe segment 16 and the ball segment 18 which are arranged adjacent thereto. Specifically, the foot orthosis 10 may be made of a thinner material in the region of the joint unit 20 and/or of a material which is more or easier bendable compared to those regions arranged adjacent thereto. Furthermore, the foot orthosis, in the region of the joint unit 20, may be made of a composite material having fibers, in particular tensile rigid fibers, which extend transversely to the pivot axis S or parallel to the longitudinal axis X of the foot orthosis. For example, the joint unit 20 may be provided in the form of a film hinge. Alternatively, the joint unit 20 may be provided in the form of a swivel joint, in particular as a single-joint hinge.

The shown foot orthosis 10 works without a metatarsal segment. However, to keep t hi a stable position on the foot to be treated which allows proper application of corrective forces, the shown configuration may be intended and configured to be worn in combination with a sock or footwear so as to provide the holding forces which contribute to the therapeutic treatment and hold the foot orthosis 10 on the foot.

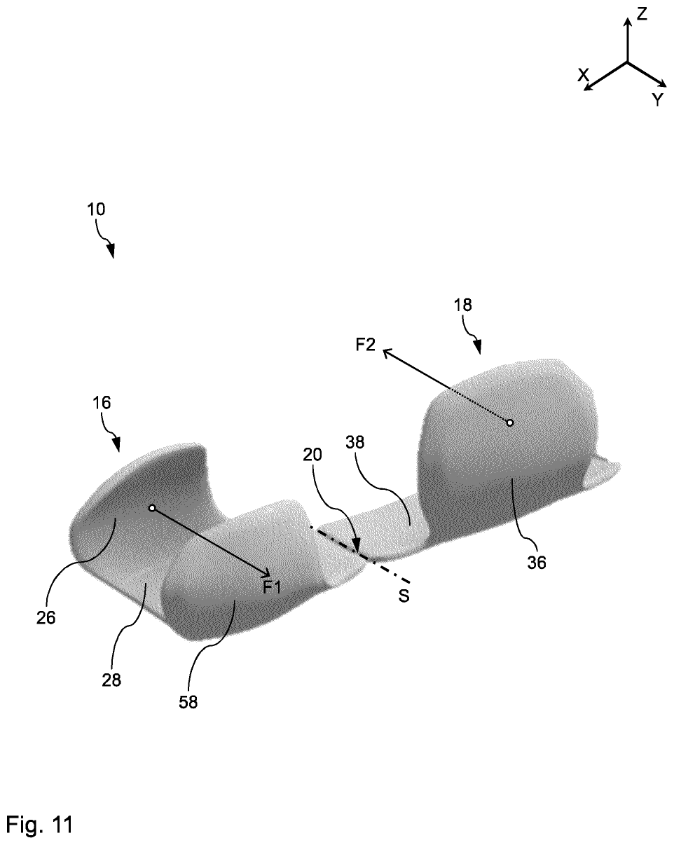

FIG. 11 shows a further embodiment of the foot orthosis 10 which, compared to the configuration shown in FIG. 10, is provided with a toe segment 16 having a medial side portion 58 which is arranged opposite the toe support section 26.

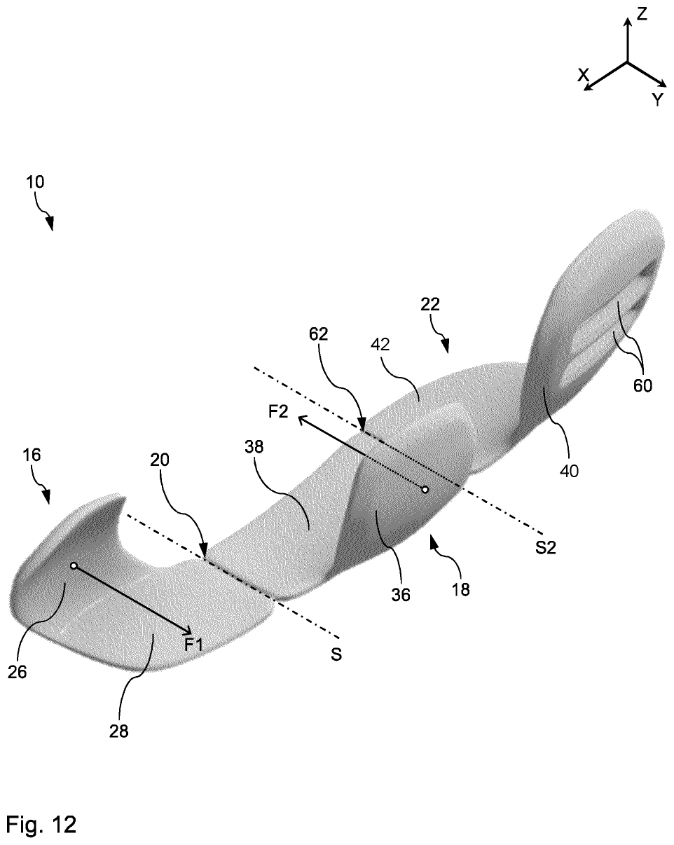

FIG. 12 shows a further embodiment of the foot orthosis 10 which, compared to the configurations shown in FIGS. 10 and 11, is provided with a metatarsal segment 22. The metatarsal segment 22 comprises a metatarsal support section 40 provided with coupling elements 60 for a support band (not shown). In the shown configuration, the coupling elements 60 are provided in the form of sit openings through which the support band is guided when being wind around the metatarsus, thereby fastening the foot orthosis 10 to the foot to be treated and applying the holding force to the foot via the support band. The metatarsal segment 22 is coupled to the ball segment 18 via a further joint unit 62 so as to be pivotable about a further pivot axis S2, which is provided parallel to the pivot axis S of the joint unit 20 between the toe segment 16 and the ball segment 18. The further joint unit 62 is designed in a corresponding manner to the joint unit 20.

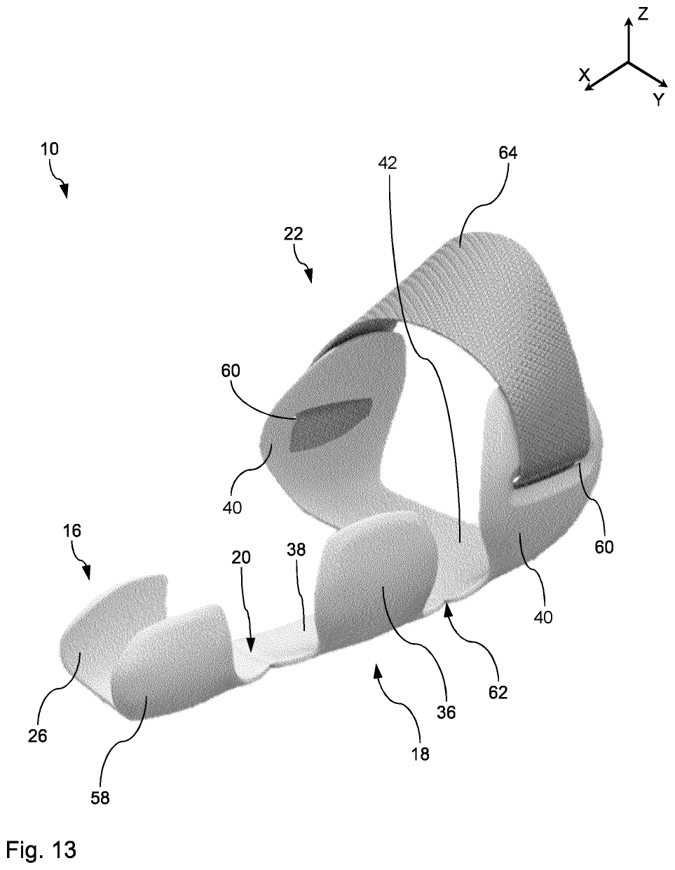

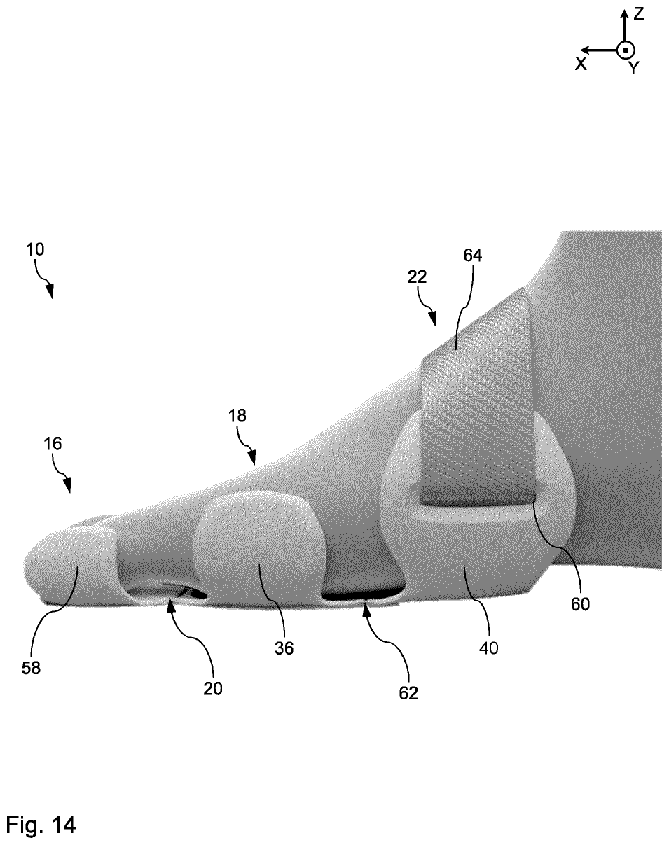

FIGS. 13 and 14 show a further embodiment of the foot orthosis 10, in which the metatarsal segment 22 comprises two opposing metatarsal support sections 40, each having a coupling element 60 through which a support band 64 is passed to be wind around the metatarsus.

Where applicable, al of the individual features illustrated in the above embodiments can be combined and/or interchanged without departing from the scope of the invention.

### LIST OF REFERENCE SIGNS

10 foot orthosis
12 big toe
14 metatarsophalangeal joint
16 toe segment
18 ball segment
20 joint unit
22 metatarsal segment
24 contact surface
26 toe support section
28 toe base section
30 clamping position of the toe segment
32 recess or through hole
34 edge of the through hole
36 ball support section
38 ball base section
40 metatarsal support section
42 metatarsal base section
44 clamping position of the metatarsal segment
46 first joint element
48 second joint element
50 receiving groove
52 connecting ring
54 connecting stud
56 foot cushion
58 side section
60 coupling element
62 further joint unit
64 support band
F1 first corrective force
F2 second corrective force
F3 holding force
L longitudinal axis of the toe
S pivot axis
S2 further pivot axis

1. A foot orthosis for correcting foot malpositions, comprising a toe segment configured to be fastened to a toe and a ball segment configured to be arranged in the region of a metatarsophalangeal joint, each of which are pivotably connected relative to one another by means of a joint unit, wherein
in a fastened state in which the foot orthosis is fastened to the foot, the foot orthosis is configured to exert a first corrective force on the toe via the toe segment and to exert a second corrective force on the metatarsophalangeal joint via the ball segment in the opposite direction to the first corrective force,
wherein the joint unit is configure such that the toe segment and the ball segment are pivotable relative to each other about pivot axis which is substantially parallel to a flexion-extension joint axis of the metatarsophalangeal joint,
wherein the toe segment is provided in the form of a bracket which, in the fastened state, at least partially engages around the toe and
wherein the toe segment comprises a toe support section which, in the fastened state lies against a lateral side of the toe.

2. The foot orthosis of claim 1, is the foot orthosis being configured such that, in the fastened state, the toe segment extends in sections along a lower side or along an upper side of the toe.

3. The foot orthosis of claim 1, wherein in the fastened state, the toe segment is configured to transmit at least one of a shearing force or a bending force both between the joint unit and the toe to be treated in direction of the first corrective force in order to generate the first corrective force.

4. The foot orthosis of claim 1, wherein the first corrective force is provided in the form of a clamping force or the bending force induced by elastic deformation of the toe segment.

5. The foot orthosis of claim 1, wherein in the fastened state, the toe segment is arranged in a clamping position in which the toe segment is elastically deflected in a direction opposite to the first corrective force relative to a rest position of the toe segment in which the toe segment is arranged in a decoupled state of the foot orthosis being decoupled from the foot.

6. The foot orthosis of claim 1, is the foot orthosis being configured such that, in the fastened state of the foot orthosis, a sideways protruding section of a metatarsophalangeal ball is received in a recess or through hole of the ball segment.

7. The foot orthosis of claim 1, wherein the second corrective force is provided in the form of a clamping force induced by an elastic deformation of the ball segment.

8. The foot orthosis of claim 1, further comprising:
a metatarsal segment configured to be fastened to the metatarsus of the foot to be treated and, in the fastened state, to exert a holding force on the metatarsus.

9. The foot orthosis of claim 1, wherein at least one of the toe segment and/or, the ball segment and/or the metatarsal segment, or any combination thereof comprises a base section and a support section introducing corrective or holding forces into the foot, wherein as compared to the base section, the support section has a lower stiffness against shear forces, bending forces, or both shear forces and bending forces in the direction of the first or second corrective force.

10. The foot orthosis of claim 1, wherein the support section is made of a material having a lower modulus of elasticity or a lower strength or a lower hardness than the base section.

11. (canceled)

12. The foot orthosis of claim 1, wherein in the fastened state, the joint unit is arranged laterally at the foot or extends along the dorsum of the foot or along the sole of the foot, and wherein the joint unit is provided in the form of a swivel joint or a bending joint.

13. The foot orthosis of claim 1, wherein the joint unit is provided in the form of a hubless swivel joint which is provided with a recess or a through hole along its pivot axis.

14. The foot orthosis of claim 6, wherein the recess or through hole has a diameter of at least 2.0 cm.

15. The foot orthosis of claim 1, wherein the ball segment and the metatarsal segment are coupled to each other such that each are translatory displaceable relative to each other, and each are fixable in a desired position relative to each other in a force-fitting manner or a form-fitting manner or both.

16. The foot orthosis of claim 1, further comprising:
a heel segment configured to be fastened to the heel of the foot to be treated and, in the fastened state, to exert a holding force on a rear part of the foot.

17. The foot orthosis of claim 8, further comprising:
a heel segment configured to be fastened to the heel of the foot to be treated and, in the fastened state, to exert a holding force on a rear part of the foot.

18. (canceled)

19. The foot orthosis according to claim 6, wherein the recess or through hole has a diameter of at least 2.5 cm.

20. The foot orthosis of claim 15, wherein the ball segment and the metatarsal segment are coupled to each other such that each are translatory displaceable relative to each other along a longitudinal axis of the foot orthosis, or along a transverse axis of the foot orthosis, or both.
